(19) United States
(12) Patent Application Publication
(10) Pub. No.: US 2019/0247014 A1
(43) Pub. Date: Aug. 15, 2019

(54) METHOD FOR OBTAINING THERMOACOUSTIC DATA

(71) Applicant: Endra Inc., Ann Arbor, MI (US)

(72) Inventors: Scott Patrick BELANGER, Ann Arbor, MI (US); Michael M. THORNTON, Ann Arbor, MI (US); Jang Hwan CHO, Ann Arbor, MI (US)

(21) Appl. No.: 15/894,100

(22) Filed: Feb. 12, 2018

(51) Int. Cl. A61B 8/08; A61B 8/00

(52) U.S. Cl. CPC A61B 8/0833; A61B 8/4494

(57) ABSTRACT

A method of obtaining thermoacoustic data comprises obtaining ultrasound data of a region of interest using at least one ultrasound transducer array, the at least one ultrasound transducer array mechanically connected to at least one thermoacoustic transducer array such that a centerline of the at least one thermoacoustic transducer array is at a known angle with respect to a centerline of the at least one ultrasound transducer array, adjusting a position of the at least one ultrasound transducer array to orient the at least one thermoacoustic transducer array in at least one imaging position based on the known angle, and obtaining thermoacoustic data of the region of interest at the at least one imaging position using the at least one thermoacoustic transducer array.

### FIELD

This application relates to imaging and in particular to a method for obtaining thermoacoustic data.

### BACKGROUND

In traditional ultrasound medical imaging, or sonography, a single array of ultrasound transducers (sometimes referred to as a transmit-receive array) operates both to transmit and receive ultrasound energy. Ultrasound transducer elements transmit ultrasound waves into an object (e.g., tissue). The transmitted energy is scattered and reflected by the tissue, and the scattered and reflected ultrasound energy is received by the same ultrasound transducer elements. The ultrasound transducer converts received ultrasound energy to ultrasound signals. The ultrasound signals are analyzed and interpreted through signal processing, generally providing information on location of structures within the tissue.

In medical ultrasound imaging, ultrasound pulses are used in a manner similar to radar, where a pulse is transmitted, and then echoes are received from reflections and from scatter within tissue. In radar (Radio Detection And Ranging), a short pulse of an electromagnetic (radiofrequency or microwave) carrier wave is transmitted, and echoes or reflections are detected by a receiver, typically co-located with the transmitter. The range of radar is limited by the received signal energy. Analogously, in ultrasound medical imaging, strong, short electrical pulses transmitted by the ultrasound system drive the transducer at a desired frequency in order to achieve good range resolution. The two-way time of flight of received echoes yields range information, and the strength of the received echoes provides information on acoustical impedance (e.g., when a transmitted pulse encounters a structure within tissue with a different density, and reflects back to the transducer). With knowledge of the direction of the transmitted pulse, an ultrasound image, or sonogram, is created. In ultrasound medical imaging, the maximum transmitted power is limited by the voltage tolerated in the system electronic components, and by the peak intensity permitted by safety considerations pertaining to tissue exposure. As in radar, the range is limited by the received signal versus background noise, which is in turn limited by total pulse energy.

Thermoacoustic imaging, sometimes referred to as photoacoustic or optoacoustic imaging, is a technology used in characterizing and imaging materials based on their electromagnetic and thermal properties, having applications in nondestructive testing, clinical diagnostics, medical imaging, life sciences and microscopy. Thermoacoustic imaging uses short pulses of electromagnetic (EM) energy, i.e., the excitation energy, to rapidly heat features within an object that absorb the EM energy (excitation sites). This rapid heating causes the material (e.g., tissue) to increase in pressure slightly, inducing acoustic pulses that radiate from the excitation site as an ultrasound wave. These acoustic pulses are detected using acoustic receivers, such as an array of ultrasound transducers, located at the material's surface. The resulting measurements are analyzed and interpreted through signal processing using time-of-flight and related algorithms, which reconstruct the distribution of absorbed EM energy, sometimes referred to as thermoacoustic computed tomography (CAT). The result can be displayed to the user as depth profile plots, or as 2-, 3-, or 4-dimensional images.

There are different requirements for clinical ultrasound transducers operating in transmit-receive mode versus receive-only ultrasound transducers employed in thermoacoustic imaging. Clinical ultrasound transducer arrays are constructed and optimized to operate in both transmit and receive ultrasound modes. These ultrasound transducers require high operating efficiency in transmitting and receiving ultrasound energy, which is not a requirement of receive-only transmitters used in thermoacoustic imaging. Clinical ultrasound transducers typically use a lens to provide an optimal depth of focus, and are designed with an optimized frequency of operation. Traditional ultrasound imaging relies upon narrow band reception for image resolution.

By contrast, in thermoacoustic imaging, it is important for the receive-only transducers to receive and process a wide band of frequencies. Thermoacoustic transducer elements and arrays are designed to operate with a high sensitivity in receive-only mode. Thermoacoustic image resolution is determined by frequency of the acoustic signal. This frequency is determined by characteristics of the material being imaged, not by the frequency of the emitted electromagnetic energy (“EM”, or excitation, energy). To be able to discriminate a range of material properties in thermoacoustic imaging (e.g., small and large size structures; imaging shallow materials and deep materials), wide reception bandwidth is important. A reception bandwidth on the order of 3-6 MHz is considered a fairly wide range, and higher bandwidths are desirable.

One consideration in image formation in both ultrasound and thermoacoustic imaging is the geometry of the transducer arrays. Different transducer geometries, such as single focused transducer, linear arrays, and two-dimensional arrays, are capable of different modes of image formation. Depending in part on the transducer geometry, the imaging system may for example image single lines, two-dimensional regions, or three-dimensional volumes. The imaging operation also may employ scanning, or motion of the transducers or transducer arrays, to adapt transducer operation to different modes of imaging.

Traditional clinical ultrasound technology identifies locations of features within a tissue or other material, but provides no functional characteristics. On the other hand, thermoacoustic imaging combines absorption contrasts achieved through interaction of the imaged material with the EM excitation energy, with fine ultrasound resolutions characteristic of acoustic reception, thereby enabling deep penetration in in vivo imaging. Thermoacoustic imaging can detect dynamic features and can measure various functional characteristics of anatomy.

Although techniques for thermoacoustic imaging have been considered, improvements are desired. It is therefore an object at least to provide a novel method for obtaining thermoacoustic data.

### SUMMARY

It should be appreciated that this Summary is provided to introduce a selection of concepts in a simplified form that are further described below in the Detailed Description. This Summary is not intended to be used to limit the scope of the claimed subject matter.

Accordingly, in one aspect there is provided a method of obtaining thermoacoustic data, the method comprising obtaining ultrasound data of a region of interest using at least one ultrasound transducer array, the at least one ultrasound transducer array mechanically connected to at least one thermoacoustic transducer array such that a centerline of the at least one thermoacoustic transducer array is at a known angle with respect to a centerline of the at least one ultrasound transducer array, adjusting a position of the at least one ultrasound transducer array to orient the at least one thermoacoustic transducer array in at least one imaging position based on the known angle, obtaining thermoacoustic data of the region of interest at the at least one imaging position using the at least one thermoacoustic transducer array.

According to another aspect there is provided a system comprising an ultrasound imaging system comprising at least one ultrasound transducer array, a thermoacoustic imaging system comprising at least one thermoacoustic transducer array mechanically connected to the at least one ultrasound transducer array such that a centerline of the at least one thermoacoustic transducer array is at a known angle with respect to a centerline of the at least one ultrasound transducer array, the at least one ultrasound transducer array moveable to orient the at least one thermoacoustic transducer array in at least one imaging position based on the known angle, and a processing unit configured to obtain thermoacoustic data of a region of interest at the at least one imaging position using the at least one thermoacoustic transducer array.

According to another aspect there is provided an imaging probe comprising at least one ultrasound transducer array, and at least one thermoacoustic transducer array mechanically connected to the at least one ultrasound transducer array such that a centerline of the at least one thermoacoustic transducer array is at a known angle with respect to a centerline of the at least one ultrasound transducer array.

### BRIEF DESCRIPTION OF THE DRAWINGS

Embodiments will now be described more fully with reference to the accompanying drawings in which:

FIG. 1 is a schematic view of an imaging system in accordance with the subject application;

FIG. 2 is a plan view of a thermoacoustic transducer mechanically connected to an ultrasound transducer;

FIG. 3 is a flow chart showing a method of obtaining thermoacoustic data;

FIG. 4 is an exemplary region of interest being imaged according to the method of FIG. 3;

FIG. 5 is a flow chart showing steps for grading an object of interest;

FIG. 6 is plan view of another embodiment of a thermoacoustic transducer mechanically connected to an ultrasound transducer;

FIG. 7 is a plan view of another embodiment of a thermoacoustic transducer mechanically connected to an ultrasound transducer;

FIG. 8 is a plan view of another embodiment of a thermoacoustic transducer mechanically connected to an ultrasound transducer;

FIG. 9 is an isometric view of another embodiment of a thermoacoustic transducer mechanically connected to an ultrasound transducer;

FIG. 10 is an isometric view of another embodiment of a thermoacoustic transducer mechanically connected to an ultrasound transducer;

FIG. 11 is an isometric view of another embodiment of a thermoacoustic transducer mechanically connected to an ultrasound transducer; and

FIG. 12 is an isometric view of another embodiment of a thermoacoustic transducer mechanically connected to an ultrasound transducer.

### DETAILED DESCRIPTION OF THE EMBODIMENTS

The foregoing summary, as well as the following detailed description of certain examples will be better understood when read in conjunction with the appended drawings. As used herein, an element or feature introduced in the singular and preceded by the word “a” or “an” should be understood as not necessarily excluding the plural of the elements or features. Further, references to “one example” or “one embodiment” are not intended to be interpreted as excluding the existence of additional examples or embodiments that also incorporate the described elements or features. Moreover, unless explicitly stated to the contrary, examples or embodiments “comprising” or “having” or “including” an element or feature or a plurality of elements or features having a particular property may include additional elements or features not having that property. Also, it will be appreciated that the terms “comprises”, “has”, “includes” means “including by not limited to” and the terms “comprising”, “having” and “including” have equivalent meanings.

As used herein, the term “and/or” can include any and all combinations of one or more of the associated listed elements or features.

It will be understood that when an element or feature is referred to as being “on”, “attached” to, “connected” to, “coupled” with, “contacting”, etc. another element or feature, that element or feature can be directly on, attached to, connected to, coupled with or contacting the other element or feature or intervening elements may also be present. In contrast, when an element or feature is referred to as being, for example, “directly on”, “directly attached” to, “directly connected” to, “directly coupled” with or “directly contacting” another element of feature, there are no intervening elements or features present.

It will be understood that spatially relative terms, such as “under”, “below”, “lower”, “over”, “above”, “upper”, “front”, “back” and the like, may be used herein for ease of description to describe the relationship of an element or feature to another element or feature as illustrated in the figures. The spatially relative terms can however, encompass different orientations in use or operation in addition to the orientation depicted in the figures.

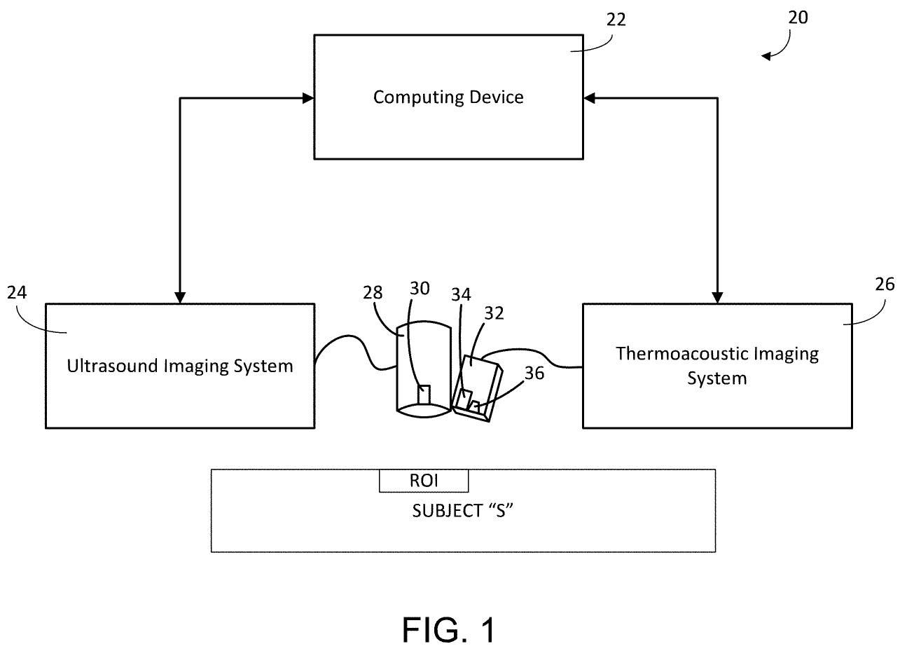

Turning now to FIG. 1, an exemplary imaging system is shown and is generally identified by reference numeral 20. As can be seen, the imaging system 20 comprises a programmed computing device 22 communicatively coupled to an ultrasound imaging system 24 and to a thermoacoustic imaging system 26. The ultrasound imaging system 24 and thermoacoustic imaging system 26 are configured to obtain ultrasound image data and thermoacoustic data, respectively, of a region of interest ROI associated with a subject S.

The programmed computing device 22 in this embodiment is a personal computer or other suitable processing device comprising, for example, a processing unit comprising one or more processors, system memory (volatile and/or non-volatile memory), other non-removable or removable memory (e.g., a hard disk drive, RAM, ROM, EEPROM, CD-ROM, DVD, flash memory, etc.) and a system bus coupling the various computer components to the processing unit. The computing device 22 may also comprise networking capabilities using Ethernet, Wi-Fi, and/or other suitable network format, to enable connection to shared or remote drives, one or more networked computers, or other networked devices. One or more input devices, such as a mouse and a keyboard (not shown) are coupled to the computing device 22 for receiving user input. A display device (not shown), such as one or more computer screens or monitors, is coupled to the computer device 22 for displaying one or more generated images that are based on ultrasound image data received from the ultrasound imaging system 24 and/or the thermoacoustic data received from thermoacoustic imaging system 26.

The ultrasound imaging system 24 comprises an ultrasound transducer 28 housing one or more ultrasound transducer arrays 30 configured to emit sound waves into the region of interest ROI of the subject S. The sound waves directed into the region of interest ROI of the subject echo off tissue within the region of interest ROI, with different tissues reflecting varying degrees of sound. These echoes are received by the one or more ultrasound transducer arrays 30 and are processed by the ultrasound imaging system 24 before being communicated as ultrasound image data to the computing device 22 for further processing and for presentation and interpretation by an operator. In this embodiment the ultrasound imaging system 24 utilizes B-mode ultrasound imaging techniques assuming a nominal speed of sound of 1,540 m/s. As ultrasound imaging systems are known in the art, further specifics of the ultrasound imaging system 24 will not be described further herein.

The thermoacoustic imaging system 26 comprises a thermoacoustic transducer 32. The thermoacoustic transducer 32 houses one or more thermoacoustic transducer arrays 34 and a radio-frequency (RF) source 36. The RF source 36 is configured to generate short pulses of RF electromagnetic radiation that are directed into the region of interest ROI of the subject to deliver energy to tissue within the region of interest ROI of the subject S. The energy delivered to the tissue induces acoustic pressure waves that are detected by the thermoacoustic imaging system 26 using the one or more thermoacoustic transducer arrays 34. In this embodiment, the RF source 36 has a frequency between about 10 Mhz and 100 Ghz and has a pulse duration between about 0.1 nanoseconds and 10 microseconds. Acoustic pressure waves detected by the one or more thermoacoustic transducer arrays 34 are processed and communicated as thermoacoustic data to the computing device 22 for further processing and for presentation and interpretation by an operator. As thermoacoustic imaging systems are known in the art, further specifics of the thermoacoustic imaging system 26 will not be described further herein.

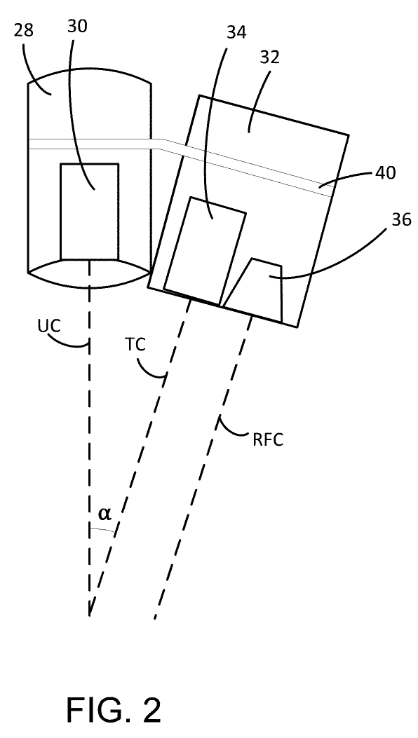

As shown in FIG. 2, the thermoacoustic transducer 32 is mechanically connected to the ultrasound transducer 28 using a connector 40. In this embodiment the connector 40 is in the form of a band or strap and is made of a rigid material such as for example metal, plastic etc. The connector 40 is extends about an outside surface of the thermoacoustic transducer 32 and the ultrasound transducer 28. In this embodiment, the connector 40 is mechanically connected to the outside surface of the thermoacoustic transducer 32 and the ultrasound transducer 28 using a fastener such as for example a screw. As will be appreciated, in other embodiments the connector 40 may be mechanically connected to the outside surface of the thermoacoustic transducer 32 and the ultrasound transducer 28 using adhesive, friction, etc.

The connector 40 is shaped such that the spatial relationship between the one or more ultrasound transducer arrays 30, the one or more thermoacoustic transducer arrays 34 and the RF source 36 is known. Put another way, the connector 40 is used to fix the spatial relationship between the one or more ultrasound transducer arrays 30, the one or more thermoacoustic transducer arrays 34 and the RF source 36. In this embodiment, the spatial relationship is set using a centerline of the one or more ultrasound transducer arrays 30, the one or more thermoacoustic transducer arrays 34, and RF source 36. Each centerline is defined as being a mid-point of an area of the respective array(s) or face.

In this embodiment, the spatial relationship between the one or more ultrasound transducer arrays 30 and the one or more thermoacoustic transducer arrays 34 is such that a thermoacoustic centerline TC of the one or more thermoacoustic transducer arrays 34 is set at known angle α with respect to an ultrasound centerline UC (also known as the axial axis or ultrasound transducer array beam axis) of the one or more ultrasound transducer arrays 30. The spatial relationship between the one or more thermoacoustic transducer arrays 34 and the RF source 36 is such that an RF centerline RFC of the RF source 36 is spaced-apart and generally parallel to the thermoacoustic centerline TC.

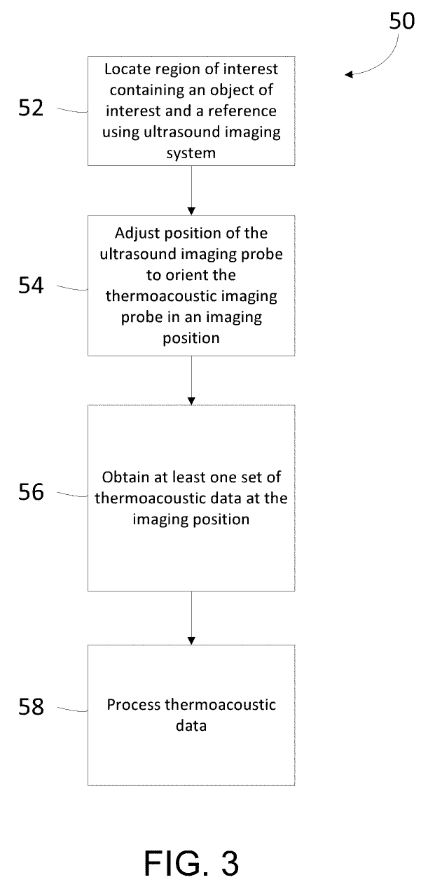

The imaging system 20 utilizes the known spatial relationship between the one or more ultrasound transducer arrays 30 and the one or more thermoacoustic transducer arrays 34 to increase the precision and accuracy of thermoacoustic imaging according to method 50, shown in FIG. 3.

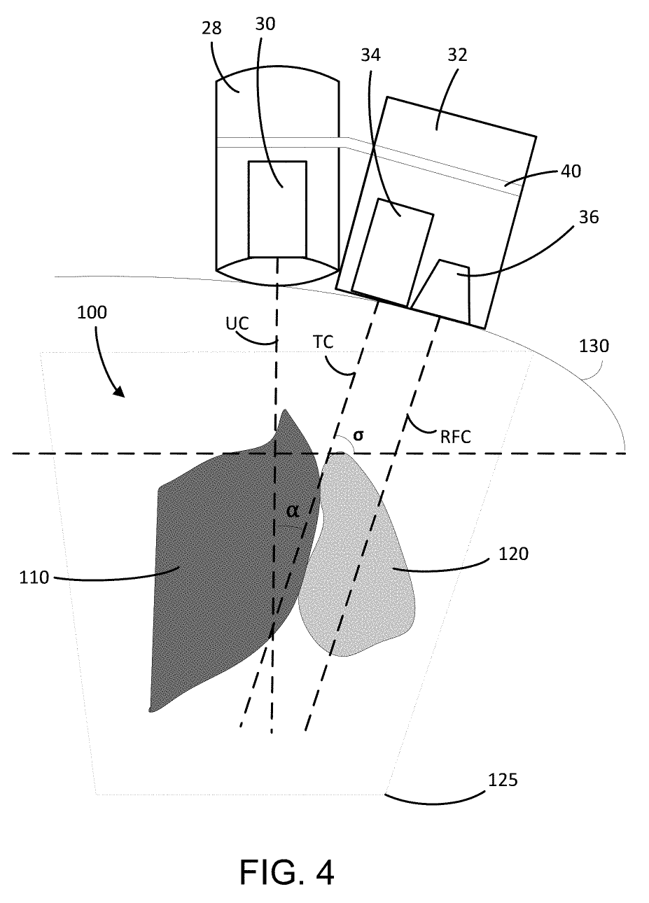

During the method 50, a region of interest is initially located within a subject's body that contains an object of interest and a reference (step 52). In this embodiment, the region of interest is located using the ultrasound imaging system 24. Specifically, ultrasound image data obtained by the ultrasound imaging system 24 is communicated to the computing device 22. The ultrasound image data is processed by the computing device 22 and a reconstructed ultrasound image is presented on the display device. The operator moves the ultrasound probe 28 on the subject's body until the region of interest is located. When locating the region of interest, the computing device 22 overlays information associated with the angle of the ultrasound centerline UC overtop of the reconstructed ultrasound image on the display device. An exemplary region of interest 100 located by the ultrasound imaging system 24 is shown in FIG. 4. The region of interest 100 contains an object of interest 110 and a reference 120. In this embodiment, the object of interest 110 is the subject's liver 110 and the reference is the subject's kidneys 120. Boundary 125 is shown in FIG. 4 for illustrative purposes only to identify an exemplary B-mode ultrasound image of the region of interest 100. The subject's skin 130 is also shown.

Since the angle α between the ultrasound centerline UC and the thermoacoustic centerline TC is known, the operator is able to adjust position of the ultrasound transducer 28 (and consequently the thermoacoustic transducer 32) with respect to the subject's body to orient the thermoacoustic transducer 32 to an imaging position (step 54). As will be appreciated, the imaging position is such that the thermoacoustic imaging system 26 is able to obtain thermoacoustic data of the region of interest at a desired imaging angle G. In the example shown in FIG. 4, the desired imaging angle G is such that the thermoacoustic centerline TC extends through a boundary between the object of interest 110 and the reference 120.

At least one set of thermoacoustic data of the region of interest is obtained at the desired imaging angle σ using the thermoacoustic imaging system 26 (step 56). The thermoacoustic imaging data is communicated to the computing device 22 for processing as desired (step 58). In the example shown in FIG. 4, the thermoacoustic data is processed to estimate the fractional fat content of the object of interest. Generally, two (2) sets of thermoacoustic data are obtained from different imaging positions. Each imaging position is chosen such that the region of interest has a different depth from the RF source 36 of the thermoacoustic imaging system 26. As will be appreciated, this ensures noticeable attenuation occurs between the two (2) sets of thermoacoustic data. The two (2) sets of thermoacoustic data are analyzed to determine the electric field strength at a boundary between the object of interest and the reference. Using the electric field strength at the boundary, the fractional fat content of the object of interest is estimated.

In this embodiment, to estimate the fractional fat content of the object of interest using the electric field strength, a number of equations are utilized. As is known, the thermoacoustic pressure produced by a heat source H(r,t) obeys the following equation:

$${∇}^{2} p ( r , t ) = \frac{1}{{c}^{2}} \frac{{\partial }^{2}}{\partial {t}^{2}} p ( r , t ) = - \frac{β}{{c}_{p}} \frac{\partial }{\partial t} H ( r , t ) \qquad [ 1 ]$$

where r is the spatial position vector, β is the isobaric volume expansion coefficient, c is the sound speed and Cₚ is the specific heat capacity. Solving equation 1 with respect to the thermoacoustic pressure yields the following forward problem:

$$p ( r , t ) = \frac{β}{4 π {C}_{p}} \int \int \int \frac{\partial r}{\vert r - {r}^{′} \vert} \frac{\partial H ( {r}^{′} , {t}^{′} )}{\partial {t}^{′}} {\vert}_{{t}^{′} = t - \frac{{r}^{′}}{C}} \qquad [ 2 ]$$

The heat source is modeled as the product of two factors: the spatial distribution of energy absorption A(r), which is the characteristics of the object of interest being imaged, and the temporal irradiation function I(t). Since the ultrasound transducer array has a finite bandwidth, the recorded thermoacoustic measurements p_{d}(r,t) are the convolution of induced pressure p(r,t) and the impulse response of the ultrasound transducer array h(t) as set out in equation 3:

p_{d}(r,t)=p(r,t)*ₜh(t) [3]

where *ₜ denotes a one-dimensional (1D) temporal convolution.

As will be appreciated, for conventional thermoacoustic imaging, the goal is to recover the absorption coefficient A(r) by inverting the forward problem. The irradiation function is modeled as a temporal function that is uniform throughout the electric field at a given time point.

Due to the limited bandwidth of the ultrasonic transducer array used to receive thermoacoustic data, accurately recovering the absorption coefficient A(r) is not trivial. As such, extracting quantitative information such as fractional fat content of the object of interest from thermoacoustic data requires sophisticated methods beyond conventional reconstruction methods.

When the object of interest is heated with an RF radiation pulse, the power deposition per unit volume is expressed as:

H(r,t)=ωε₀εᵣ″E²(r,t) [4]

where ω is the radian frequency, ε₀ is the vacuum permittivity, εᵣ″ is the relative permittivity of the tissue and E(r,t) is the electric field strength. The strength of thermoacoustic data obtained from a tissue is the product of the absorption coefficient of the tissue μₐ, the deposited energy W(r,t), and the Gruneisen parameter of the tissue, Γ:

S(r,t)=μₐΓW(r,t)=μₐΓωε₀εᵣ″E²(r,t) [5]

As will be appreciated, because of the impulse response characteristic of the ultrasound transducer, the recorded thermoacoustic data exhibits bipolar signals at a boundary between two different tissues. The strength of the bipolar thermoacoustic data is defined as a distance between two peaks of the bipolar signal. As will be appreciated, in other embodiments other information such as for example a width of the bimodal signal may be incorporated.

Within lossy medium, the electric field strength is attenuated as it propagates through the medium. The amount of attenuation is determined by characteristics of the medium, and is modeled as shown in equation 6:

Loss(d)=e^{-ηd} [6]

where η is the attenuation coefficient of the medium.

The electric field strength is expressed as a function of depth as shown in equation 7:

E(d)=E₀Loss(d)=E₀e^{-∫}^{₀}^{^{d}}^{η(x)dx} [7]

As will be appreciated, in other embodiments different models may be used.

In this embodiment, equation 5 is used as a model to infer fractional fat content from the thermoacoustic data. As mentioned previously, thermoacoustic data obtained from the boundary between the object of interest and the reference is a bipolar signal. The strength of the bipolar signal represents the absorption property difference between the object of interest and the reference. Further, the phase of the thermoacoustic data at the boundary indicates which tissue (object of interest or the reference) has a higher or lower absorption coefficient. The strength of the thermoacoustic data at the boundary is expressed in equation 8:

S_{bipolar}(r,t)=(μ_{a,1}Γ₁ε_{0,1}ε_{r,1}′−μ_{a,2}Γ₂ε_{0,2}ε_{r,2}″)ωE²(r,t), [8]

where subscripts 1 and 2 denote two different tissues.

As shown in equation 8, the strength of the acquired thermoacoustic data is determined by several tissue properties and the strength of the electric field.

The strength of each set of thermoacoustic data is different due to the attenuation of the electric field between the different imaging positions. Since the object of interest is located between the two imaging positions, the attenuation of the electric field is characterized by the dielectric properties of the object of interest, which are associated with the fractional fat content of the liver. Using equations 7 and 8, the ratio of the strength of the thermoacoustic data at the object of interest to the strength of the thermoacoustic data at the reference can be expressed as:

S_{bipolar,Object of Interest}/S_{bipolar,Reference}=E₀²e^{-η}^{ₜᵢₛₛᵤₑ}^{d}, [9]

where E₀ is the electric field strength at the RF source of the thermoacoustic imaging system 26, d is the distance between the positions of where the two (2) sets of thermoacoustic data are obtained. Since the properties of the reference are known, to estimate the fractional fat content of the object of interest, only the strength of the electric field at the boundary is required. Put another way, since tissue with a higher fractional fat content will have different dielectric and thermal properties than lean (no fat content) tissue, the fractional fat content of the region of interest of a tissue ηₜᵢₛₛᵤₑ is deduced.

Further details on how to estimate the fractional fat content of the object of interest may be found in U.S. patent application Ser. Nos. 15/666,535 and 15/666,546, both entitled “A Method and System for Estimating Fractional Fat Content of an Object”, the relevant portions of which are incorporated herein by reference.

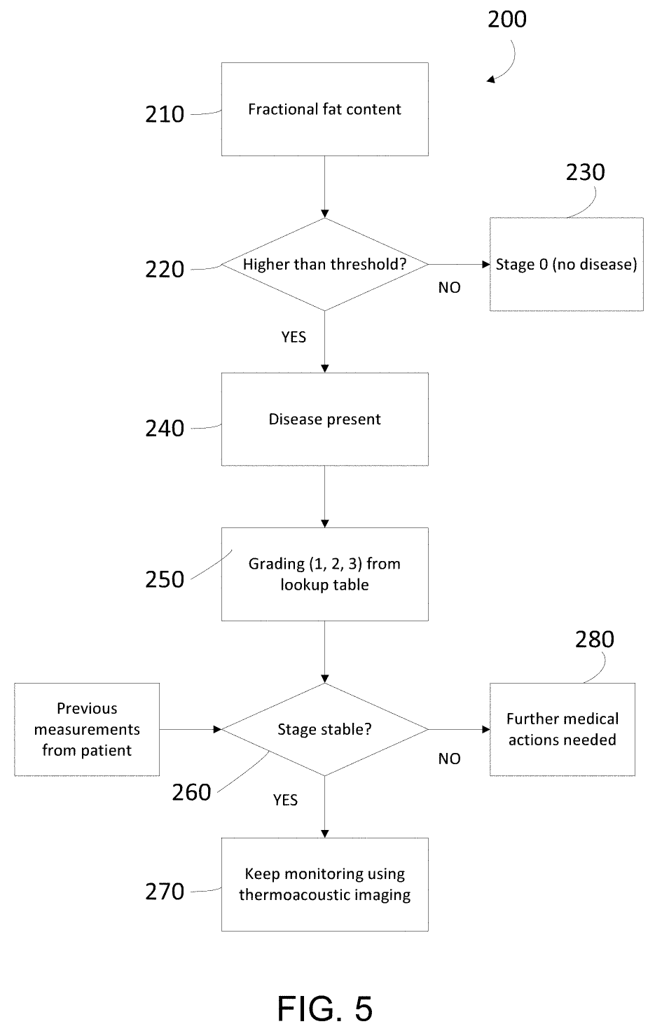

The object of interest is graded using the estimated fractional fat content. An exemplary method 200 of grading the object of interest using the estimated fractional fat content is shown in FIG. 5. During the method, the estimated fractional fat content (step 210) is compared to a threshold (step 220). In this embodiment, the threshold is for fatty liver disease and is set at a fractional fat content of 5%. Specifics of the threshold for fatty liver disease are outlined in “Magnetic resonance imaging and liver histology as biomarkers of hepatic steatosis in children with non-alcoholic fatty liver disease,” authored by Schwimmer, Hepatology, vol. 61, pp. 1887-1895, 2015.

If the estimated fractional fat content is less than the threshold, it is determined that the subject does not have liver disease and thus the object of interest is graded as a zero (0) (step 230). If the estimated fractional fat content is higher than the threshold, it is determined that a disease such as steatosis is present (step 240). The object of interest is in turn graded as a one (1), two (2) or three (3) by comparing the estimated fractional fat content to known tabulated values (step 250). In this embodiment, the known tabulated values are outlined in “Non-alcoholic steatohepatitis: A proposal for grading and staging the histological lesions,” authored by Brunt et al., Am. J. Gastroenterol., vol. 94, no. 9, pp. 2467-2474, September 1999.

Specifically, in this embodiment, the object of interest is graded as a one (1) if the estimated fractional fat content is between 5% and 33%. The object of interest is graded as a two (2) if the estimated fractional fat content is between 34% and 66%. The object of interest is graded as a three (3) if the estimated fractional fat content is greater than 66%.

The grade of the object of interest is then compared to previous grades obtained for the subject (if available) (step 260). If the grade of the object of interest has not changed, the object of interest is deemed stable and periodic monitoring using thermoacoustic imaging is recommended (step 270). If the grade of the object of interest has changed, further medical actions are deemed to be required (step 280).

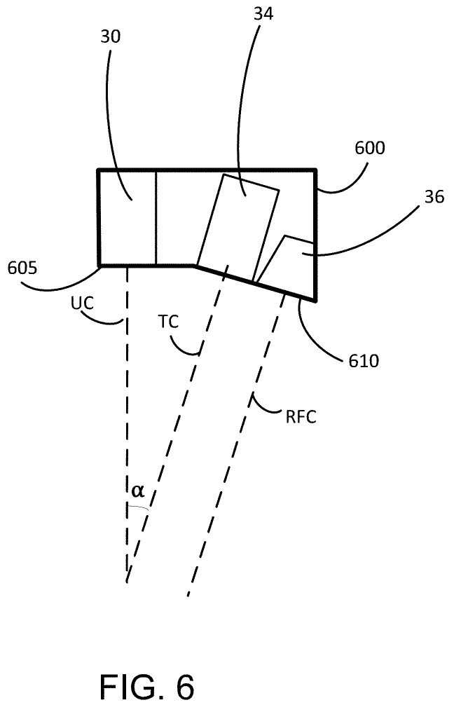

Although in embodiments the thermoacoustic transducer 32 is mechanically connected to the ultrasound transducer 28 using a connector, those skilled in the art will appreciate that alternatives are available. Turning now to FIG. 6, another embodiment is shown. In this embodiment, a housing 600 is mechanically connect the one or more ultrasound transducer arrays 30, the one or more thermoacoustic transducer arrays 34 and the RF source 36. In this embodiment, the housing 600 comprises a generally flat surface 605 and an angled surface 610. The angled surface 610 extends from the generally flat surface 605 at a known angle. The one or more ultrasound transducer arrays 30 are mechanically connected to the housing 600 adjacent the generally flat surface 605. The one or more thermoacoustic transducer arrays 34 and the RF source 36 are mechanically connected to the housing 600 adjacent the angled surface 610. As such, the spatial relationship between the one or more ultrasound transducer arrays 30, the one or more thermoacoustic transducer arrays 34 and the RF source 36 is fixed. An exterior surface of the housing 600 may include features such as indents or finger pads to enhance comfort for the user. In another embodiment, the housing 600 may comprise connectors such as fasteners, clips or straps configured to receive the one or more ultrasound transducer arrays 30, the one or more thermoacoustic transducer arrays 34 and the RF source 36. In this embodiment, the one or more ultrasound transducer arrays 30, the one or more thermoacoustic transducer arrays 34 and the RF source 36 may be removably connected to the housing 600. In another embodiment, the housing, the one or more ultrasound transducer arrays, the one or more thermoacoustic transducer arrays and the RF source form a dual-purpose imaging probe. In this embodiment, the dual-purpose imaging probe may be connectable to an existing ultrasound imaging system and an existing thermoacoustic imaging system. In another embodiment, the one or more thermoacoustic transducer arrays and the RF source may be mechanically connected to the housing. In this embodiment, the housing may comprise an opening mechanism configured to provide access to the housing to removably receive one or more ultrasound transducer arrays of an existing ultrasound imaging system. In another embodiment, the housing may comprise an opening mechanism configured to provide access to the housing to receive the one or more thermoacoustic transducer arrays and the RF source of an existing thermoacoustic imaging system and to receive one or more ultrasound transducer arrays of an existing ultrasound imaging system. In an embodiment, the housing may comprise two segments that are positionable around the one or more ultrasound transducer arrays, the one or more thermoacoustic transducer arrays and the RF source. In this embodiment, the two segments may be connectable via snap-fit to form the housing. In other embodiments, the housing may not be opened.

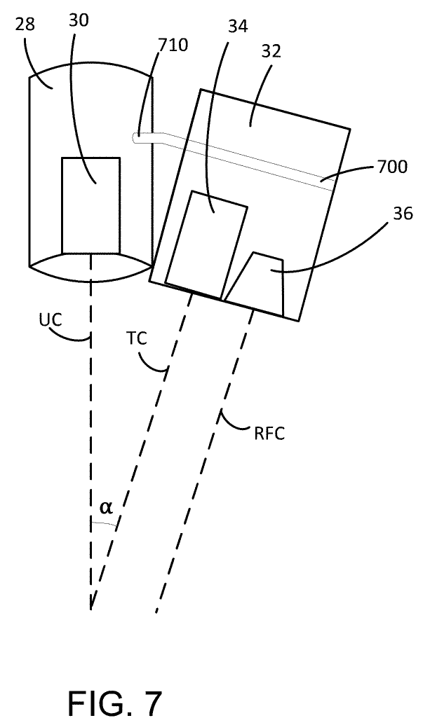

Another embodiment is shown in FIG. 7. As can be seen, connector 700 is generally similar to that of connector 40, with the following exceptions. In this embodiment, connector 700 is mechanically connected to the thermoacoustic transducer 32. The connector 700 extends from the thermoacoustic transducer 32 and comprises a U-shaped portion 710. The U-shaped portion 710 is dimensioned to receive the ultrasound transducer 28 via friction fit. As will be appreciated, the dimensions of the U-shaped portion are such that one or more particular types or models of ultrasound transducers may be received therein. As will be appreciated, the U-shaped portion may be of any shape to receive an ultrasound transducer via friction fit. As will be appreciated, in another embodiment, rather than being mechanically connected to the thermoacoustic transducer, the connector may be mechanically connected to the ultrasound transducer and may receive a thermoacoustic transducer via friction fit. In another embodiment, the connector may comprise a first portion configured to receive the ultrasound transducer via friction fit and a second portion configured to receive the thermoacoustic transducer via friction fit.

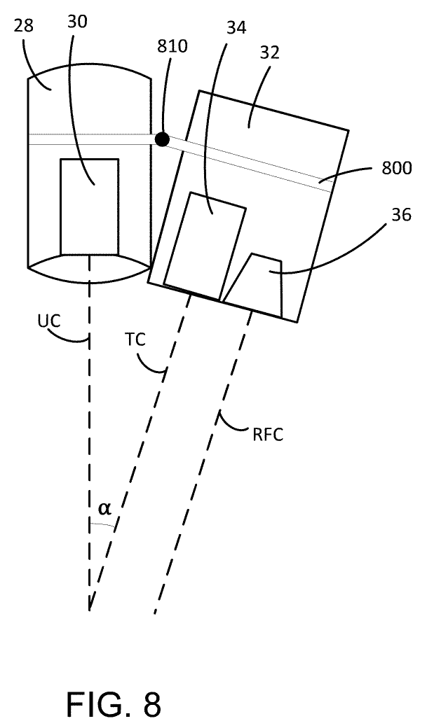

Another embodiment is shown in FIG. 8. As can be seen, connector 800 is generally similar to that of connector 40, with the following exceptions. In this embodiment, connector 800 comprises an adjustment mechanism 810 located at a position intermediate the ultrasound transducer 28 and the thermoacoustic transducer 32. In this embodiment, the adjustment mechanism 810 comprises a threaded screw and is rotatable to adjust the angle α between the thermoacoustic centerline TC of the one or more thermoacoustic transducer arrays 34 and the ultrasound centerline UC of the one or more ultrasound transducer arrays 30. As will be appreciated, the adjustment mechanism 810 enables the connector 800 to be used with different types or models of ultrasound transducers and/or thermoacoustic transducers. As will be appreciated, other types of adjustment mechanisms may be used.

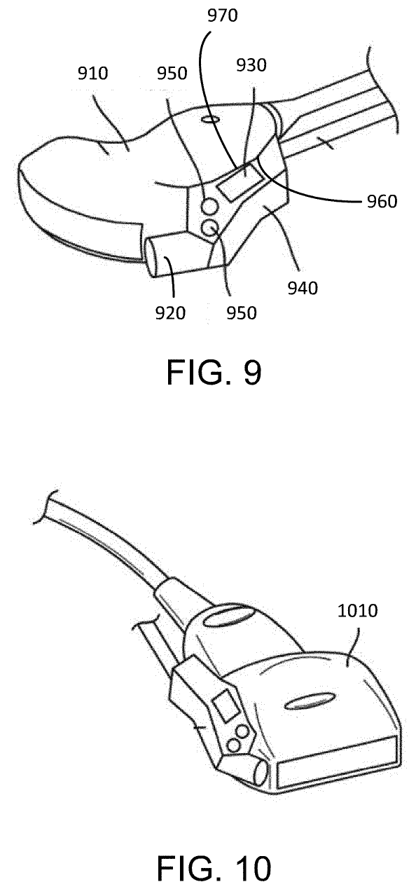

Although in embodiments the thermoacoustic transducer is mechanically connected to the ultrasound transducer using a connector, those skilled in the art will appreciate that alternatives are available. Turning now to FIG. 9, another embodiment is shown. In this embodiment, rather than mechanically connecting the thermoacoustic transducer to the ultrasound transducer using a connector, the thermoacoustic transducer 920 is mechanically connected to the ultrasound transducer 910 using a fastening device (not shown). A display screen 930 is positioned on a housing 940 of the thermoacoustic transducer 920. Indicators 950 are positioned on the housing 940 and are used to indicate an imaging status of the imaging system.

The ultrasound transducer 910 is of a known type. In this embodiment, the ultrasound transducer 910 is a curvilinear ultrasound transducer such as for example a General Electric C1-6-D ultrasound transducer.

The display screen 930 is used to display the known angle to a user. As will be appreciated, in other embodiments the display screen 930 may be used to display additional or alternative data such as for example thermoacoustic measurement data.

The housing 940 is made of a rigid material such as for example biocompatible plastic. In this embodiment, the housing 940 is dimensioned and shaped such that a surface 960 of the housing 940 complements a surface 970 of the ultrasound transducer 910 and, when the thermoacoustic transducer 920 is connected to the ultrasound transducer 910, the thermoacoustic centerline is at the known angle with respect to the ultrasound centerline. The housing 940 of the thermoacoustic transducer 920 is mechanically connected to the ultrasound transducer 910 using a fastening device (not shown) at a position on surfaces 960 and 970. The fastening device may be a screw, adhesive, tape, glue, etc.

Another embodiment is shown in FIG. 10. The embodiment shown in FIG. 10 is generally identical to that of the embodiment shown in FIG. 9, with the following exception. In this embodiment, the ultrasound transducer 1010 is in the form of a linear ultrasound transducer such as for example a General Electric 8-L ultrasound transducer.

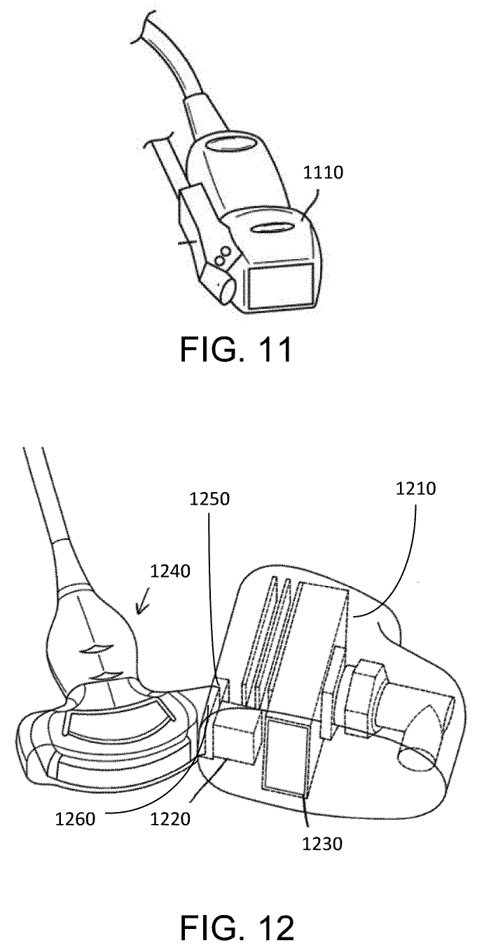

Another embodiment is shown in FIG. 11. The embodiment shown in FIG. 11 is generally identical to that of the embodiment shown in FIG. 9, with the following exception. In this embodiment, the ultrasound transducer 1110 is in the form of a phased ultrasound transducer such as for example a General Electric 12S-D ultrasound transducer.

Although in embodiments the thermoacoustic transducer is mechanically connected to the ultrasound transducer using a connector, those skilled in the art will appreciate that alternatives are available. Turning now to FIG. 12, another embodiment is shown. In this embodiment, rather than mechanically connecting the thermoacoustic transducer to the ultrasound transducer using a connector, a housing 1210 is used to house the thermoacoustic transducer 1220 and the RF source 1230. The housing 1210 is connectable to the ultrasound transducer 1240.

The ultrasound transducer 1240 is of a known type. In this embodiment, the ultrasound transducer 1240 is a curvilinear ultrasound transducer such as for example a General Electric C1-6-D ultrasound transducer.

In this embodiment, the housing 1210 is made of a rigid material such as for example biocompatible plastic. The housing 1210 is made a number of pieces that, when assembled, form the housing 1210. The thermoacoustic transducer 1220 and RF source 1230 are mechanically secured within the housing 1210 using structural members (not shown) located within the housing 1210.

The housing 1210 comprises an opening 1250 dimensioned and shaped to receive a portion 1260 of the ultrasound transducer 1240 via friction fit. The opening 1240 is dimensioned and shaped based on known dimensions of the ultrasound transducer 1250.

Although in embodiments described above the computing device is described as being connected to the ultrasound imaging system and the thermoacoustic imaging system, those skilled in the art will appreciate that alternatives are available. For example, in another embodiment the computing device may act as both the thermoacoustic and ultrasound imaging systems. In this embodiment, the computing device is connected to the ultrasound transducer and the thermoacoustic transducer and receives ultrasound and thermoacoustic imaging data, respectively, therefrom.

Although in embodiments the desired imaging angle G is described as being such that the thermoacoustic centerline TC extends through a boundary between the object of interest 110 and the reference 120, those skilled in the art will appreciate that alternatives are available. For example, in another embodiment the desired imaging angle σ is between 0 degrees and 90 degrees. In another embodiment, the desire desired imaging angle σ is such that the thermoacoustic centerline TC and the ultrasound centerline UC cross at a boundary between the object of interest and the reference. In another embodiment, the desired imaging angle σ is such that the thermoacoustic centerline TC and the ultrasound centerline UC cross within the object of interest.

Although in embodiments described above, two sets of thermoacoustic data are acquired to estimate the fractional fat content of the object of interest, those skilled in the art will appreciate that alternatives are available. For example, in another embodiment, only one set of thermoacoustic data may be required. In this embodiment, it is assumed that a subject having a high fractional fat content of an object of interest, such as the liver, will have a thick layer of subcutaneous fat. The subcutaneous fat may be used as the reference to estimate the fractional fat content of the liver. In this embodiment, the electric field strength at the boundary between the object of interest (liver) and the reference (subcutaneous fat) is estimated using the subcutaneous fat. In this embodiment, ultrasound image data is processed to estimate the thickness of the subcutaneous fat. Using the thickness of the subcutaneous fat, the set of thermoacoustic data is analyzed to determine the electric field strength after the subcutaneous fat. Using equation 8 (above), properties related to the object of interest (liver) are calculated. The calculated properties may be translated to fractional fat content by comparing the properties to tabulated data.

As will be appreciated, in the above embodiments, thermoacoustic signal strength received by an ultrasound transducer array may be affected by various factors that are not related to signal generation, but rather associated with acoustic propagation. These factors depend on transducer spatial sensitivity, relative positioning between the ultrasound transducer array and the boundary between the object of interest and the reference, and the relative shape of the reference with respect to the ultrasound transducer array surface. Even for the same subject and the same ultrasound transducer array, changing the position and angle of the ultrasound transducer array during thermoacoustic data acquisition results in different measurements.

To compensate for these factors, one or more compensation factors may be calculated. The one or more compensation factors may be based on information and measurements provided by the user or estimated using ultrasound image data. Each factor may be calculated information such as size and shape of the reference and the angle between the ultrasound transducer array and the boundary. In an embodiment, the one or more compensation factors may be calculated based on theoretical methods such as by using acoustic propagation and ultrasound transducer properties. In another embodiment, the one or more compensation factors may be obtained from phantom and clinical studies. In yet another embodiment, both theoretical and experimental methods may be used.

The above embodiments may include an image correction method, such as that described in above-incorporated U.S. patent application Ser. Nos. 15/666,535 and 15/666,546. Generally, the image correction method may comprise determining the translational and elevational angle between the ultrasound transducer array and the tangent line of the boundary. A check is performed to determine if the reference is a blood vessel. If the reference is a blood vessel, such as a portal vein, the shape of the blood vessel is estimated by the computing device using ultrasound image data or known segmentation methods and the method continues. As will be appreciated, the shape of the blood vessel may be the thickness of the blood vessel, the length of the blood vessel within the field of view, and the cross section of the blood vessel. If the reference is not a blood vessel, the method does not perform this step. A compensation factor is calculated using information obtained in the previous steps and using ultrasound transducer array characteristics. The information that is used to calculate the compensation factor may be at least one of the translational angle, the elevational angle, the thickness of the blood vessel (if selected as the reference), and spatial sensitivity of the ultrasound transducer array. Once the compensation factor has been calculated, thermoacoustic data is adjusted using the compensation factor.

Although in embodiments described above the region of interest is described as comprising an object of interest, a reference, and a boundary between the object of interest and the reference, those skilled in the art will appreciate that alternatives are available. For example, in another embodiment, the region of interest may contain an object of interest, a reference and an intermediate structure in between the reference and the object of interest. In this embodiment, a boundary between the reference and the intermediate structure is identified. The electric field strength at the intermediate structure is estimated using obtained thermoacoustic data. With the electric field strength of the intermediate structure known, the intermediate structure is used as a new reference. A boundary between the new reference and the object of interest is identified. The fractional fat content of the object of interest is then estimated using the electric field strength of the new reference.

Those skilled in the art will appreciate that the above-described ultrasound image data and thermoacoustic data may be one-dimensional, two-dimensional or three-dimensional. In embodiments, the ultrasound image data may be in a different dimension than the thermoacoustic data. For example, ultrasound image data may be two-dimensional and the thermoacoustic data may be one-dimensional.

Those skilled in the art will appreciate that other objects of interest may be evaluated and other references may be used such as for example the heart, kidney(s), lung, esophagus, thymus, breast, prostate, brain, muscle, nervous tissue, epithelial tissue, bladder, gallbladder, intestine, liver, pancreas, spleen, stomach, testes, ovaries, uterus, skin and adipose tissues.

Other aspects of the subject application are exemplified in the following clauses:

A1. A method of obtaining thermoacoustic data, the method comprising:

obtaining ultrasound data of a region of interest using at least one ultrasound transducer array, the at least one ultrasound transducer array mechanically connected to at least one thermoacoustic transducer array such that a centerline of the at least one thermoacoustic transducer array is at a known angle with respect to a centerline of the at least one ultrasound transducer array;

adjusting a position of the at least one ultrasound transducer array to orient the at least one thermoacoustic transducer array in at least one imaging position based on the known angle;

obtaining thermoacoustic data of the region of interest at the at least one imaging position using the at least one thermoacoustic transducer array.

A2. The method of clause A1, wherein the obtaining thermoacoustic data of the region of interest comprises:

directing pulses of radio-frequency (RF) electromagnetic radiation into the region of interest using an RF source.

A3. The method of clause A2, wherein the RF source is mechanically connected to the at least one thermoacoustic transducer array.
A4. The method of clause A3, wherein a centerline of the RF source is generally parallel to the centerline of the at least one thermoacoustic transducer array.
A5. The method of clause A1, further comprising:

locating the region of interest using the at least one ultrasound transducer array.

A6. The method of clause A1, further comprising:

adjusting the known angle prior to obtaining the ultrasound data of the region of interest.

A7. The method of clause A1, wherein the at least one imaging position is a preferred imaging angle between the centerline of the at least one thermoacoustic transducer array and the region of interest.
A8. The method of clause A1, wherein the at least one imaging position is selected to include at least one object of interest within the region of interest.
A9. The method of clause A1, wherein the at least one imaging position is selected to include at least one object of interest and at least one reference within the region of interest.
A10. The method of clause A9, further comprising:

processing the thermoacoustic data to estimate fractional fat content of the object of interest.

A11. The method of clause A10, wherein processing the thermoacoustic data to estimate fractional fat content of the object of interest comprises:

determining an electric field strength between the object of interest and the at least one reference.

A12. The method of clause A10, further comprising:

obtaining thermoacoustic data of the region of interest at least at two imaging positions using the at least one thermoacoustic transducer array.

A13. The method of clause A10 further comprising:

grading the object of interest using the estimated fractional fat content.

A14. The method of clause A13, wherein the object of interest is a liver.
A15. The method of clause A1, further comprising:

mechanically connecting the at least one ultrasound transducer array to the at least one thermoacoustic transducer array at the known angle prior to obtaining ultrasound data.

A16. The method of clause A15, wherein the mechanically connecting comprises securing the at least one ultrasound transducer array and the at least one thermoacoustic transducer array to a connector.
A17. The method of clause A15, wherein the mechanically connecting comprises securing one of the at least one ultrasound transducer array and the at least one thermoacoustic transducer array to a connector and mechanically connecting the other of the at least one ultrasound transducer array and the at least one thermoacoustic transducer array to the connector using a snap-fit connection.
A18. The method of clause A15, further comprising:

adjusting the known angle.

A19. The method of clause A18, wherein the adjusting the known angle is based on a type of at least one of the at least one ultrasound transducer array and the at least one thermoacoustic transducer array.
A20. The method of clause A15, wherein the mechanically connecting comprises securing the at least one ultrasound transducer array and the at least one thermoacoustic transducer array within a single housing.
A21. The method of clause A1, wherein the known angle is between 45 degrees and 90 degrees.
B1. A system comprising:

an ultrasound imaging system comprising at least one ultrasound transducer array;

a thermoacoustic imaging system comprising at least one thermoacoustic transducer array mechanically connected to the at least one ultrasound transducer array such that a centerline of the at least one thermoacoustic transducer array is at a known angle with respect to a centerline of the at least one ultrasound transducer array, the at least one ultrasound transducer array moveable to orient the at least one thermoacoustic transducer array in at least one imaging position based on the known angle; and

a processing unit configured to:
obtain thermoacoustic data of a region of interest at the at least one imaging position using the at least one thermoacoustic transducer array.
   B2. The system of clause B1, further comprising a connector mechanically connecting the at least one at least one thermoacoustic transducer array to the at least one ultrasound transducer array.
   B3. The system of clause B2, wherein the connector comprises an adjustment mechanism configured to adjust the known angle prior to imaging.
   B4. The system of clause B1, wherein the connector comprises a U-shaped portion dimensioned to mechanically connect to at least one of the at least one ultrasound transducer array and the at least one thermoacoustic transducer array via friction fit.
   B5. The system of clause B1, further comprising an ultrasound transducer comprising the at least one ultrasound transducer array.
   B6. The system of clause B1, further comprising a thermoacoustic transducer comprising the at least one thermoacoustic transducer array.
   B7. The system of clause B1, further comprising a housing configured to house the at least one thermoacoustic transducer array and the at least one ultrasound transducer array.
   B8. The system of clause B1, wherein the thermoacoustic imaging system comprises a radio-frequency (RF) source.
   B9. The system of clause B8, wherein the RF source is mechanically connected to the at least one thermoacoustic transducer array.
   B10. The system of clause B9, wherein a centerline of the RF source is generally parallel to the centerline of the at least one thermoacoustic transducer array.
   B11. The system of clause B8, further comprising a thermoacoustic transducer comprising the at least one thermoacoustic transducer array and the RF source.
   B12. The system of clause B11, wherein a centerline of the RF source is generally parallel to the centerline of the at least one thermoacoustic transducer array.
   B13. The system of clause B1, wherein the known angle is between 45 degree and 90 degrees.
   B14. The system of clause B1, wherein the at least one imaging position is a preferred imaging angle between the centerline of the at least one thermoacoustic transducer array and the region of interest.
   B15. The system of clause B1, wherein the at least one imaging position is selected to include at least one object of interest within the region of interest.
   B16. The system of clause B1, wherein the at least one imaging position is selected to include at least one object of interest and at least one reference within the region of interest.
   B17. The system of clause B16, wherein the processing structure is further configured to:

processing the thermoacoustic data to estimate fractional fat content of the object of interest.

B18. The system of clause B17, wherein the processing structure is further configured to:

grade the object of interest using the estimated fractional fat content.

C1. An imaging probe comprising:

at least one ultrasound transducer array; and

at least one thermoacoustic transducer array mechanically connected to the at least one ultrasound transducer array such that a centerline of the at least one thermoacoustic transducer array is at a known angle with respect to a centerline of the at least one ultrasound transducer array.

C2. The imaging probe of clause C1 comprising:

a connector mechanically connecting the at least one at least one thermoacoustic transducer array to the at least one ultrasound transducer array.

C3. The imaging probe of clause C2, wherein the connector comprises an adjustment mechanism configured to adjust the known angle.
C4. The imaging probe of clause C2, wherein the connector comprises a U-shaped portion dimensioned to mechanically connect to at least one of the at least one ultrasound transducer array and the at least one thermoacoustic transducer array via friction fit.
C5. The imaging probe of clause C1, further comprising an ultrasound transducer comprising the at least one ultrasound transducer array.
C6. The imaging probe of clause C1, further comprising a thermoacoustic transducer comprising the at least one thermoacoustic transducer array.
C7. The imaging probe of clause C1, further comprising a housing configured to house the at least one thermoacoustic transducer array and the at least one ultrasound transducer array.
C8. The imaging probe of clause C1, further comprising a radio-frequency (RF) source.
C9. The imaging probe of clause C8, wherein the RF source is mechanically connected to the at least one thermoacoustic transducer array.
C10. The imaging probe of clause C9, wherein a centerline of the RF source is generally parallel to the centerline of the at least one thermoacoustic transducer array.
C11. The imaging probe of clause C8, further comprising a thermoacoustic transducer comprising the at least one thermoacoustic transducer array and the RF source.
C12. The imaging probe of clause C11, wherein a centerline of the RF source is generally parallel to the centerline of the at least one thermoacoustic transducer array.
C13. The imaging probe of clause C1, wherein the known angle is between 45 degree and 90 degrees.

Although embodiments have been described above with reference to the accompanying drawings, those of skill in the art will appreciate that variations and modifications may be made without departing from the scope thereof as defined by the appended claims.

What is claimed is:

1. A method of obtaining thermoacoustic data, the method comprising:
obtaining ultrasound data of a region of interest using at least one ultrasound transducer array, the at least one ultrasound transducer array mechanically connected to at least one thermoacoustic transducer array such that a centerline of the at least one thermoacoustic transducer array is at a known angle with respect to a centerline of the at least one ultrasound transducer array;
adjusting a position of the at least one ultrasound transducer array to orient the at least one thermoacoustic transducer array in at least one imaging position based on the known angle; and
obtaining thermoacoustic data of the region of interest at the at least one imaging position using the at least one thermoacoustic transducer array.

2. The method of claim 1, wherein the obtaining thermoacoustic data of the region of interest comprises:
directing pulses of radio-frequency (RF) electromagnetic radiation into the region of interest using an RF source.

3. The method of claim 2, wherein the RF source is mechanically connected to the at least one thermoacoustic transducer array.

4. The method of claim 3, wherein a centerline of the RF source is generally parallel to the centerline of the at least one thermoacoustic transducer array.

5. The method of claim 1, further comprising:
locating the region of interest using the at least one ultrasound transducer array.

6. The method of claim 1, further comprising:
adjusting the known angle prior to obtaining the ultrasound data of the region of interest.

7. The method of claim 1, wherein the at least one imaging position is a preferred imaging angle between the centerline of the at least one thermoacoustic transducer array and the region of interest.

8. The method of claim 1, wherein the at least one imaging position is selected to include at least one object of interest within the region of interest.

9. The method of claim 1, wherein the at least one imaging position is selected to include at least one object of interest and at least one reference within the region of interest.

10. The method of claim 9, further comprising:
processing the thermoacoustic data to estimate fractional fat content of the object of interest.

11. The method of claim 10, wherein processing the thermoacoustic data to estimate fractional fat content of the object of interest comprises:
determining an electric field strength between the object of interest and the at least one reference.

12. The method of claim 10, further comprising:
obtaining thermoacoustic data of the region of interest at least at two imaging positions using the at least one thermoacoustic transducer array.

13. The method of claim 10 further comprising:
grading the object of interest using the estimated fractional fat content.

14. The method of claim 13, wherein the object of interest is a liver.

15. The method of claim 1, further comprising:
mechanically connecting the at least one ultrasound transducer array to the at least one thermoacoustic transducer array at the known angle prior to obtaining ultrasound data.

16. The method of claim 15, wherein the mechanically connecting comprises securing the at least one ultrasound transducer array and the at least one thermoacoustic transducer array to a connector.

17. The method of claim 15, wherein the mechanically connecting comprises securing one of the at least one ultrasound transducer array and the at least one thermoacoustic transducer array to a connector and mechanically connecting the other of the at least one ultrasound transducer array and the at least one thermoacoustic transducer array to the connector using a snap-fit connection.

18. The method of claim 15, further comprising:
adjusting the known angle.

19. The method of claim 18, wherein the adjusting the known angle is based on a type of at least one of the at least one ultrasound transducer array and the at least one thermoacoustic transducer array.

20. The method of claim 15, wherein the mechanically connecting comprises securing the at least one ultrasound transducer array and the at least one thermoacoustic transducer array within a single housing.

21. The method of claim 1, wherein the known angle is between 45 degrees and 90 degrees.
